(19) United States
(12) Patent Application Publication
(10) Pub. No.: US 2005/0187302 A1
(43) Pub. Date: Aug. 25, 2005

(54) Method for treatment or prevention of osteoporosis in individuals with high bone turnover

(75) Inventors: Taru Blom, Nousiainen (FI); Lauri Kangas, Lieto (FI); Risto Lammintausta, Turku (FI)

Correspondence Address: ROTHWELL, FIGG, ERNST & MANBECK, P.C., 1425 K STREET, N.W., SUITE 800, WASHINGTON, DC, 20005, US

(73) Assignee: Hormos Medical Corporation, Turku (FI)

(21) Appl. No.: 10/783,092

(22) Filed: Feb. 23, 2004

(51) Int. Cl. A61K 31/075

(52) U.S. Cl. 514/720

(57) ABSTRACT

This invention relates to a method for the treatment or prevention of osteoporosis in an individual suffering from increased bone turnover, said method comprising administering to said individual an effective amount of a therapeutically active compound, which is a selective estrogen receptor modulator of the triphenylalkene or triphenylalkane structure.

### FIELD OF THE INVENTION

This invention relates to a method for treatment or prevention of osteoporosis in individuals with high bone turnover by administering an effective amount of a selective estrogen receptor modulator of triphenylalkane or triphenylalkene structure, particularly ospemifene or a geometric isomer, a stereoisomer, a pharmaceutically acceptable salt, an ester thereof or a metabolite thereof.

### BACKGROUND OF THE INVENTION

The publications and other materials used herein to illuminate the background of the invention, and in particular, cases to provide additional details respecting the practice, are incorporated by reference.

Bone is constantly being rebuilt throughout life in a process of bone remodeling. The remodeling begins with resorption (degradation) of bone by osteoclasts. The resorbed bone is then replaced by new bone tissue, which is characterized by collagen formation by osteoblasts, and subsequent calcification of the tissue. In healthy young adults the overall rate of remodeling is in balance, i.e. the amount of bone lost is approximately equal to the amount formed. Osteoporosis is a chronic, progressive condition, where the balance is shifting towards higher resorption than formation. Therefore, the amount of bone decreases and the bones become fragile. Osteoporosis is ofter called “the silent disease”, because bone loss occurs without any symptoms until the bone fracture. The term “osteoporosis” is commonly considered simply in terms of the amount of bone present in the body. However, WHO and consensus development conferences recommend the definition “Osteoporosis is a disesase characterized by low bone mass and microarchitectural deterioration of bone tissue, leading to enhanced bone fragility and a consequent increase in fracture risk” (Consensus development conference: diagnosis, prophylaxis and treatment of osteoporosis, American Journal of Medicine (1991) 90:107-110; Report of a WHO study group, WHO Technical Repost Series 843: Assessment of fracture risk and its application to screening for menopausal osteoporosis).

The degradation and formation cycle of the bone is called bone turnover. High turnover is found e.g. in children, but it can follow also by drugs (e.g. by corticosteroids) and bone diseases like osteomalacia. High turnover generally means both rapid bone formation and rapid bone degradation. In children high turnover is necessary as the bones grow. In elderly the bone turnover decreases and the bone mass begins to decrease. Steroid hormones are important factors in bone turnover. Their role is seen clearly in the elderly. In women the decrease of estrogen levels is considered to be the main reason to bone loss. Therefore estrogens are commonly used to protect against osteoporosis. As estrogens increase the risk of breast and uterine cancers, selective estrogen receptor modulators (SERMs) have been introduced as effective drugs in prevention and treatment of osteoporosis. The mechanism of action of SERMs is mainly to decrease the number of osteoclasts. Therefore, the bone resorption is decreased and the bone amount is maintained. SERMs and estrogens have relatively weak effects on osteoblasts.

The development of osteoporosis can be followed by measuring the bone mineral density and amount of bone in the body at certain intervals. There are also biochemical bone markers, which are specific for bone formation and bone degradation. They can be analysed either from serum (s in the table below) or in urine (u). Such markers include e.g.

| For bone formation | For bone resorption |
|---|---|
| Total alkaline phosphatase(s) | Tartrate-resistant acid phosphatase |
| Osteocalcin (s) | especially its subtype 5b (TRAP5b) (s) |
| Procollagen type1 | Total and dialyzable hydroxyproline (u) |
| N-terminal peptide (s) | Pyridinoline and |
| Procollagen type 1 | deoxypyridinoline |
| C-terminal peptide (s) | (collagen cross-links) (u) |
| | Crosslaps (s) |
| | Type 1 collagen telopeptides (u) |

At high bone turnover both formation and resorption markers may be increased, but high levels of resorption markers when compared to formation markers may also indicate high turnover in short run.

SERMs have both estrogen-like and antiestrogenic properties (Kauffman & Bryant, 1995). The effects may be tissue-specific as in the case of tamoxifen and toremifene which have estrogen-like effects in the bone, partial estrogen-like effect in the uterus and liver, and pure antiestrogenic effect in breast cancer. Raloxifene and droloxifen are similar to tamoxifen and toremifene, except that their antiestrogenic properties dominate. Based on the published information, many SERMs are more likely to cause menopausal symptoms than to prevent them. They have, however, other important benefits in elderly women: they decrease total and LDL cholesterol, thus deminishing the risk of cardiovascular diseases, and they may prevent osteoporosis and inhibit breast cancer growth in postmenopausal women. There are also almost pure antiestrogens under development.

Ospemifene is the Z-isomer of the compound of formula (I) 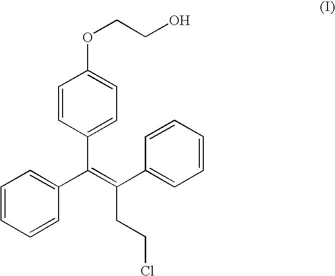
and it is one of the main metabolites of toremifene, is known to be an estrogen agonist and antagonist (Kangas, 1990; International patent publications WO 96/07402 and WO 97/32574). The compound is also called (deaminohydroxy)toremifene and it is also known under the code FC-1271a. Ospemifene has relatively weak estrogenic and antiestrogenic effects in the classical hormonal tests (Kangas, 1990). It has anti-osteoporosis actions and it decreases total and LDL cholesterol levels in both experimental models and in human volunteers (International patent publications WO 96/07402 and WO 97/32574). It also has antitumor activity in an early stage of breast cancer development in an animal breast cancer model. Ospemifene is also the first SERM which has been shown to have beneficial effects in climacteric syndromes in healthy women. The use of ospemifene for the treatment of certain climacteric disorders in postmenopausal women, namely vaginal dryness and sexual dysfunction, is disclosed in WO 02/07718. The published patent application WO 03/103649 describes the use of ospemifene for inhibition of atrophy and for the treatment or prevention of atrophy-related diseases or disorders in women, especially in women during or after the menopause.

### OBJECT AND SUMMARY OF THE INVENTION

An object of the present invention is to provide a particular subgroup of individuals especially benefiting from the administration of a SERM of triphenylalkane or triphenylalkene structure, especially ospemifene or a geometric isomer, a stereoisomer, a pharmaceutically acceptable salt, an ester thereof or a metabolite thereof in the treatment or prevention of osteoporosis.

Thus, the invention concerns a method for the treatment or prevention of osteoporosis in an individual suffering from increased bone turnover, said method comprising administering to said individual an effective amount of a therapeutically active compound, which is a selective estrogen receptor modulator of triphenylalkene or triphenylalkane structure.

### BRIEF DESCRIPTION OF THE DRAWINGS

FIG. 1A shows the individual changes in the bone resorption marker U-NTX (nmol/mmol) Crea (Creat=Creatine) with a 90 mg daily dose of ospemifene in a 12-week clinical study for several individuals.

FIG. 1B shows the individual changes in the bone resorption marker U-NTX (nmol/mmol) Crea with a 60 mg daily dose of ospemifene in a 12-week clinical study for several individuals.

FIG. 2 shows the individual changes in the bone formation marker S-PICP (microgram/l) with a 90 mg daily dose of ospemifene in a 12-week clinical study for several individuals.

FIG. 3 is a plotter chart of individual changes in the bone formation marker S-PINP (microgram/l) at 12 weeks compared to baseline in a clinical study on ospemifene for several individuals. 30=30 mg daily dose of ospemifene; 60=60 mg daily dose of ospemifene; 90=90 mg daily dose of ospemifene and 0=placebo.

FIG. 4 is a plotter chart of individual changes in the bone resorption marker U-CTX at 12 weeks compared to baseline in a clinical study on ospemifene for several individuals. 30=30 mg daily dose of ospemifene; 60=60 mg daily dose of ospemifene; 90=90 mg daily dose of ospemifene and 0=placebo.

### DETAILED DESCRIPTION OF THE INVENTION

Suitable SERM compounds for use in the present invention are triphenylalkene or triphenylalkane compounds such as compounds disclosed in WO 01/36360, U.S. Pat. No. 4,996,225, U.S. Pat. No. 4,696,949, U.S. Pat. No. 5,750,576, WO 99/42427 and the toremifene metabolites disclosed in L. Kangas, Cancer Chemother Pharmacol (1990) 27:8-12. As examples of specific drugs disclosed in the aforementioned references can be mentioned toremifene and ospemifene. Tamoxifen and its derivatives such as 4-hydroxytamoxifen, alpha-hydroxytamoxifen, N-desmethyltamoxifen, N,N-didesmethyltamoxifen, deaminotamoxifen, and droloxifene and iodoxifene also examples of suitable SERMs of triphenylalkene structure.

According to preferred embodiment, the therapeutically active compound is a SERM of triphenylalkene structure. Especially a compound of formula (I) or a geometric isomer, a stereoisomer, a pharmaceutically acceptable salt, an ester thereof or a metabolite thereof is preferred:

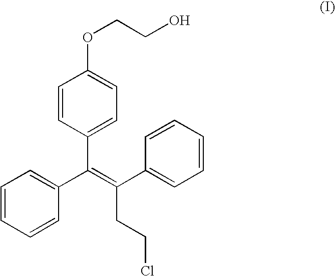

The method of preventing or treating osteoporosis with ospemifene and related compounds according to this invention in individuals with increased bone turnover is particularly useful when treating women during or after the menopause. However, the method according to this invention is not restricted to women in this age group.

The term “metabolite” shall be understood to cover any ospemifene or (deaminohydroxy)toremifene metabolite already discovered or to be discovered. As examples of such metabolites can be mentioned the oxidation metabolites mentioned in Kangas (1990) on page 9 (TORE VI, TORE VII, TORE XVIII, TORE VIII, TORE XIII), especially TORE VI and TORE XVIII, and other metabolites of the compound. The most important metabolite of ospemifene is 4-hydroxyospemifene, which has the formula 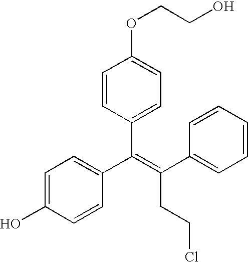
The use of mixtures of isomers of compound (I) shall also be included in this invention.

The wording “increased bone turnover” means that both bone resorption and formation of new bone are increased. As a normal value for bone resorption in postmenopausal women is considered a bone resorption of at least 65 nmol/mmol Crea, using amino terminal telopeptide of type I collagen measured in urine (U-NTX) as marker or at least 680 microgram/mmol Crea, using carboxy terminal telopeptide of type I collagen measured in urine (U-CTX) as marker. As a normal value for bone formation in the same group is considered a bone formation of at least 170 microgram/I, using carboxy terminal propeptide of type I procollagen measured in serum (S-PICP) as marker, or at least 84 microgram/I, using amino terminal propeptide of type I procollagen measured in serum (S-PINP) as marker.

A particular good response to the administering of ospemifene is observed in individuals with at least 5%, preferably at least 10% increased bone turnover, measured as well as bone resorption as bone formation.

An especially important population benefiting from the method according to this invention is postmenopausal women having a bone resorption, measured as U-NTX, which is at least 70 nmol/mmol Crea, preferably at least 80 nmol/mmol, and a bone formation, measured as S-PICP, being at least 180 microgram/I.

Particularly suitable markers for measuring bone resorption are Crosslaps measured from serum and TRAP5b, also measured from serum. Crosslaps is marker reporting the activity of osteoclasts and TRAP5b is a marker revealing the number of the osteoclasts. The value indicating a level of normal bone turnover for both of these markers is about 3. Increased bone resorption is often registered as value 6, i.e. an increase of 100%. These markers are thus very sensitive to changes in bone resorption.

According to a particularly preferred alternative, the bone resorption is measured using as markers a combination of Crosslaps and TRAP5b, both measured from serum.

According to previous data, the optimal clinical dose of ospemifene is expected to be higher than 25 mg daily and lower than 100 mg daily. A particularly preferable daily dose has been suggested in the range 30 to 90 mg. At the higher doses (100 and 200 mg daily), ospemifene shows properties more similar to those of tamoxifen and toremifene.

The invention will be disclosed more in detail in the following non-restrictive Experimental Section.

### EXPERIMENTAL SECTION

In female rats high bone turnover can be induced by ovariectomy (OVX). Rapidly, within days after OVX the number of osteoclasts increases and resorption markers increase. Shortly after OVX the bone formation is also increased, but due to the absence of bone protecting estrogens, the balance is towards bone loss. The bone loss, however, reaches within a few months a new balance, where the bone mass is lower than at baseline, but the rates of formation and resorption are equal. Estrogens can prevent the bone loss effectively, when it is administered immediately after OVX. If the administration is started months later, the bone structure has been changed and estrogens do not have as strong beneficial effect.

### EXPERIMENTAL

High bone turnover was induced to 2-4 months old female rats by ovariectomy (OVX). Treatment with ospemifene was started at different time points after OVX: 1 day, 1, 2, and 3 months after OVX. Bone resorption was evaluated in short-term by bone specific TRAP5b, which is a protease secreted specifically by osteoclasts, and later by pyridinoline/deoxypyridinoline cross links, which are degradation products of bone collagen and excreted in the urine. Finally, during autopsy, usually after 3 months treatment, trabecular bone mineral density was measured.

### RESULTS

1. Short-term Effects of Ospemifene

Female rats, 2 months old, n=6 in each group, were ovariectomized. During OVX a blood sample was taken to measure the base line value for TRAP5b. TRAPb-values were measured also after 2 days. The working hypothesis was that OVX increases TRAP5b concentrations at 2 days, because osteoclast number rapidly increases. On the other hand ospemifene was expected to lower the osteoclast number and thus decrease the TRAP5b at 2 days. As shown in Table 1, this was the case. The beneficial effect of ospemifene at the time of high bone turnover is thus obvious.

TABLE 1
Change of TRAP5b activity from baseline 2 days after OVX.
Dose of ospemifene 10 mg/kg. TRAP5b is marker of high turnover.

| | Change of TRAP5b |
|---|---|
| | activity in serum (%) |
| | 2 days after OVX |
| OVX Control (vehicle) (n = 6) | +16.1 ± 7.2 |
| OVX + Ospemifene (n = 6) | −23.3 ± 5.8 |

2. Efficacy of Ospemifene at Different Time Points after OVX

Female rats, age about 4 months, were ovariectomized. Treatment with vehicle or ospemifene was started at different periods after OVX (from one day to 3 months). The ospemifene doses were 5, 10 or 25 mg/kg and the treatment period was 3 months. Evaluation of the bone occurred by bone mineral density measurement of tibial trabecular bone after the treatment and in some groups also by bone collagen degradation products excreted in the urine.

The results are presented in Tables 2 and 3.

TABLE 2
Bone mineral density (BMD mg/cm²)
in tibial trabecular bone of rats treated with vehicle or ospemifene.
The treatment was started at different time points after OVX.

| | Trabecular BMD mg/cm² | | | |
|---|---|---|---|---|
| | at different time points after OVX | | | |
| | 1 day | 1 month | 2 months | 3 months |
| | (n = 20) | (n = 8) | (n = 8) | (n = 8) |
| Sham-operated rats | 420 ± 18 | 290 ± 38 | 291 ± 56 | 276 ± 33 |
| Ovx rats | 236 ± 13 | 90 ± 27 | 69 ± 17 | 77 ± 11 |
| OVX + Ospemifene | 374 ± 21 | | | |
| 5 mg/kg | | | | |
| OVX + Ospemifene | 392 ± 15 | | | |
| 25 mg/kg | | | | |
| OVX + Ospemifene | | 138 ± 24 | 78 ± 14 | 78 ± 17 |
| 10 mg/kg | | | | |

1 day after OVX, when the bone turnover is very high, ospemifene is able to prevent almost completely the OVX-induced bone loss

1 month after OVX, when the bone turnover is high, but lower than immediately after OVX, ospemifene has significant beneficial bone effect

2 months after OVX, when the bone turnover is markedly decreased, ospemifene has modest beneficial bone effect

3 months after OVX, when the bone turnover is low, ospemifene has almost lost the benefial bone effect

TABLE 3
Effect of ospemifene on bone degradation markers 30 days after
OVX. The administration of ospemifene was started one day after
OVX and continued daily until measurements. Urine total
pyridinoline/deoxypyridoline crosslinks were measured in the urine.
Sham means rats, which were operated like OVX animals,
but the ovaries were not removed.

| | | | OVX + Ospemifene | |
|---|---|---|---|---|
| | OVX | Sham | 5 mg/kg | 25 mg/kg |
| Change from base line | 165 ± 42 | 3 ± 22 | 55 ± 16 | 33 ± 20 |
| at 30 days | | | | |

A strong increase in crosslinks during the first month after OVX (during high bone turnover) is evident. Ospemifene significantly decreases the excretion of crosslinks, which is a resorption marker.

In the clinical trials, bone turnover was evaluated by measuring the levels of bone formation markers in serum and bone resorption markers in urine. Both bone resorption markers e.g. amino terminal telopeptide of type I collagen (U-NTX) and carboxy terminal telopeptide of type I collagen (U-CTX) and formation markers e.g. amino terminal propeptide of type I procollagen (S-PINP) and carboxy terminal propeptide of type I procollagen (S-PICP) are increased in menopause, indicating high bone turnover. Bone antiresorptive therapy decreases these values reflecting inhibition of bone turnover.

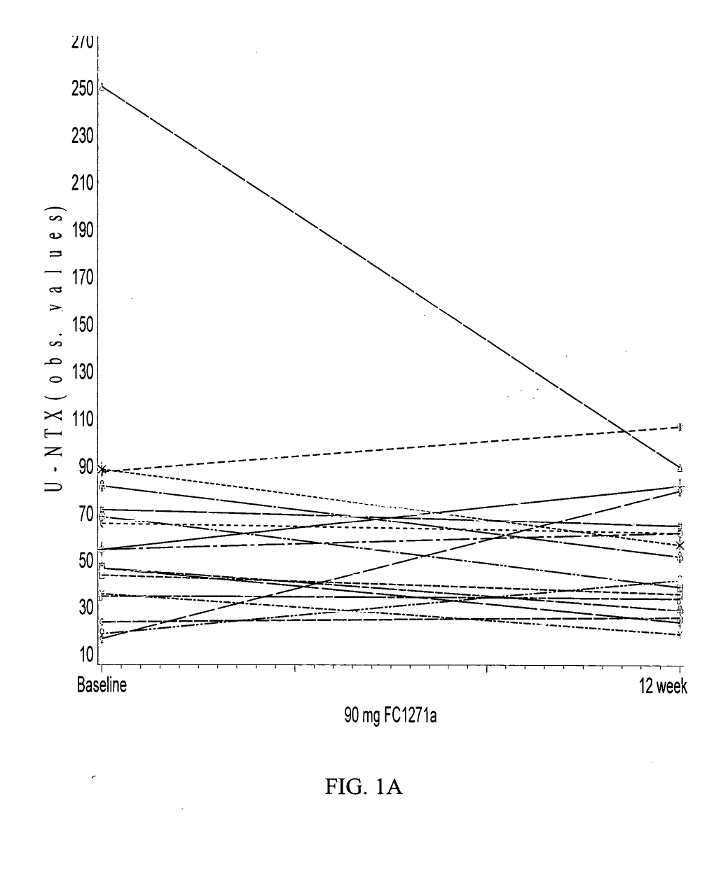

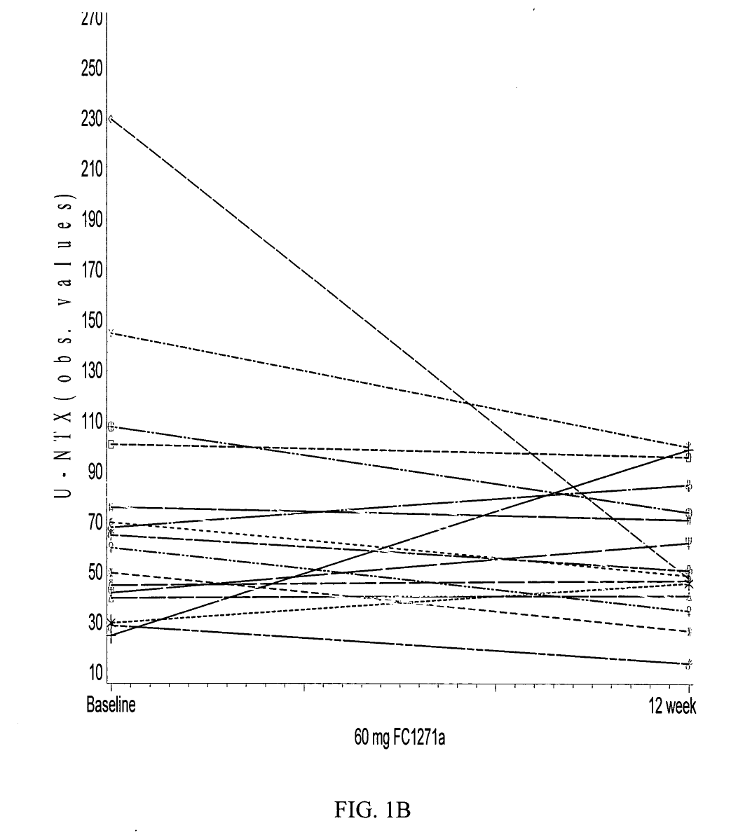

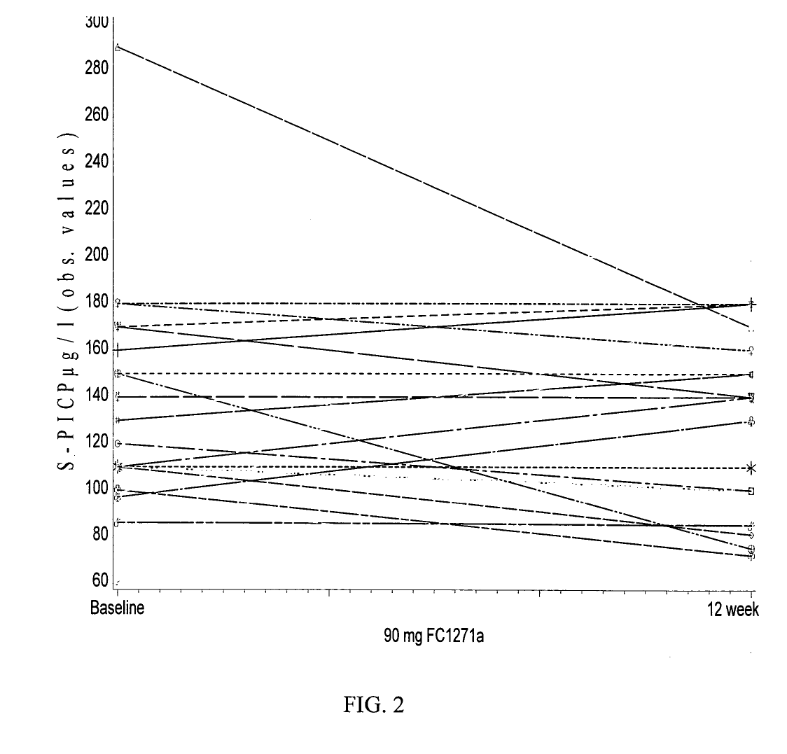

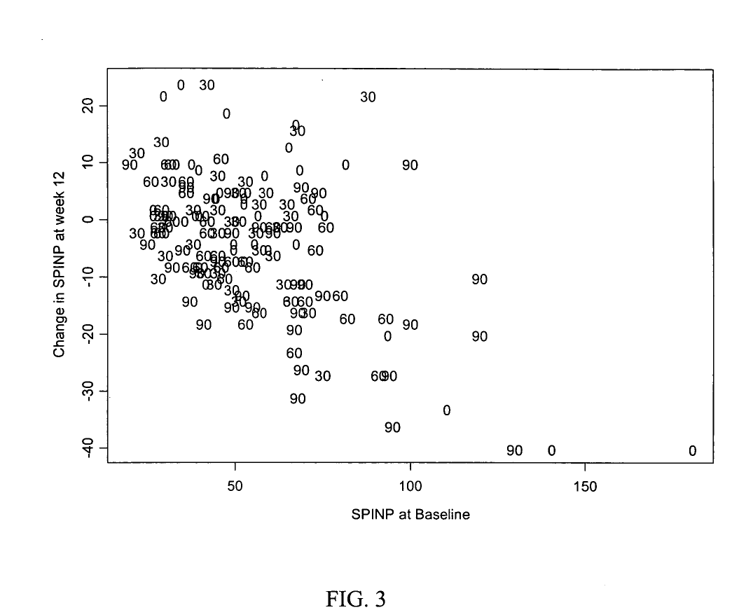

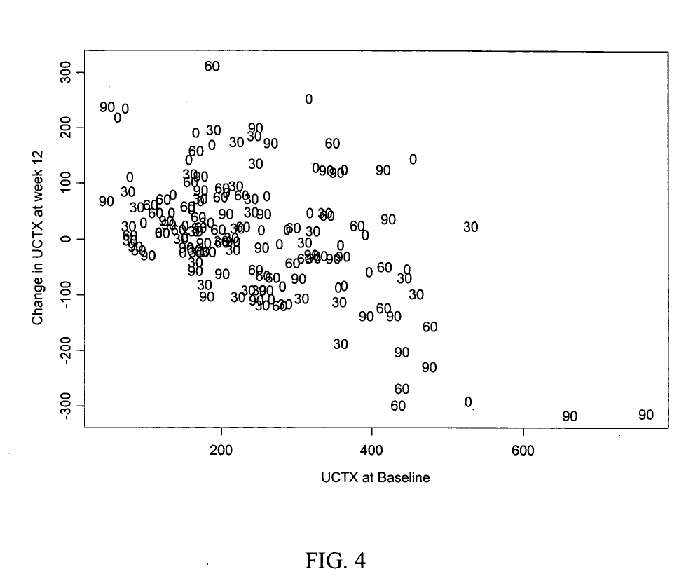

In the two 12-week phase II studies, 209 postmenopausal women were treated with 30 mg, 60 mg or 90 mg ospemifene per day. Most of the women had normal bone marker levels at baseline. In those who had high bone marker levels at baseline a large decrease both in formation and resorption bone markers was seen with daily doses 60 mg and 90 mg. As examples, individual changes in U-NTX and S-PICP are shown in FIGS. 1 and 2. In FIGS. 3 and 4, changes at 12 weeks relative to baseline in the bone markers were plotted versus the corresponding baseline values in the placebo-controlled phase II study. Large baseline values resulted in large reductions in the primary endpoints. This tendency was obvious for the formation markers S-PINP and S-PICP as well as for the resorption markers U-CTX and U-NTX in subjects treated with 60 mg or 90 mg daily doses. The upper limit of normal range in women was 84 μg/l for S-PINP, 65 nmol/mmol Crea for U-NTX, 170 μg/l for S-PICP and 680 μg/mmol Crea for U-CTX. In women with the highest bone marker levels at baseline the decrease was most dramatic.

### CONCLUSIONS

The clinical tests show that administration of ospemifene is useful in decreasing bone turnover in individuals with increased bone turnover. It is known that other drugs, such as bisphosphonates very effectively reduce bone resorption as a result of inactivation of osteoclasts. However, such a complete inactivation of osteoclasts has an adverse effect on the formation of new bone, because osteoclasts are important to eliminate old bone so that new bone can be created. Therefore, prolonged bisphosphonate treatment tends to result in a very brittle bone structure. Ospemifene has a gentle effect on the osteoclasts in that it decreases the number of the cells but it does not cause complete inactivation of the same. Therefore, ospemifene decreases the bone resorption to a certain extent, but it allows the osteoclasts to work and therefore new bone to be formed. The result is a balanced decrease in bone resorption which does not adversely affect the bone formation.

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

### REFERENCES

Kangas L. Biochemical and pharmacological effects of toremifene metabolites. Cancer Chemother Pharmacol 27:8-12, 1990.

Kauffinan R F, Bryant H U. Selective estrogen receptor modulators. Drug News Perspect 8:531-539, 1995.

1. A method for the treatment or prevention of osteoporosis in an individual suffering from increased bone turnover, said method comprising administering to said individual an effective amount of a therapeutically active compound, which is a selective estrogen receptor modulator of triphenylalkene or triphenylalkane structure.

2. The method according to claim 1 wherein the therapeutically active compound is a compound of the formula (I) 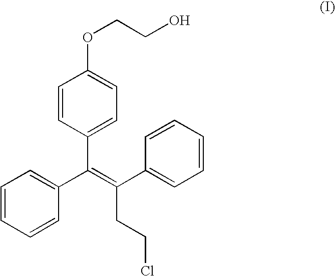
or a geometric isomer, a stereoisomer, a pharmaceutically acceptable salt, an ester thereof or a metabolite thereof.

3. The method according to claim 2 wherein compound (I) is ospemifene.

4. The method according to claim 1, wherein the individual is a postmenopausal woman.

5. The method according to claim 1, wherein the increased bone turnover is a bone resorption and a bone formation being at least 5%, preferably at least 10% higher than the normal values for these markers.

6. The method according to claim 1 wherein the individual has
a) a bone resorption of at least 65 nmol/mmol Creatine, using amino terminal telopeptide of type I collagen measured in urine (U-NTX) as marker, and/or at least 680 microgram/mmol Creatine, using carboxy terminal telopeptide of type I collagen measured in urine (U-CTX) as marker, and
b) a bone formation of at least 170 microgram/l, using carboxy terminal propeptide of type I procollagen measured in serum (S-PICP) as marker and/or at least 84 microgram/l, using amino terminal propeptide of type I procollagen measured in serum (S-PINP) as marker.

7. The method according to claim 6 where the bone resorption, measured as U-NTX, is at least 70 nmol/mmol Creatine, and the bone formation, measured as S-PICP, is at least 180 microgram/l.

8. The method according to claim 7 where the bone resorption, measured as U-NTX, is at least 80 nmol/mmol Creatine.

9. The method according to claim 5 wherein the bone resorption has been measured wherein the bone resorption has been measured using as marker Crosslaps measured from serum.

10. The method according to claim 5 wherein the bone resorption has been measured using as marker TRAP5b measured from serum.

11. The method according to claim 5 wherein the bone resorption has been measured using as markers a combination of Crosslaps and TRAP5b, both measured from serum.
